(19) United States
(12) Patent Application Publication
(10) Pub. No.: US 2007/0020339 A1
(43) Pub. Date: Jan. 25, 2007

(54) COMPOSITIONS AND METHODS FOR CONTROLLING ABUSE OF MEDICATIONS

(75) Inventor: David M. Bear, Wellesley, MA (US)

Correspondence Address: SUTHERLAND ASBILL & BRENNAN LLP, 999 PEACHTREE STREET, N.E., ATLANTA, GA, 30309, US

(73) Assignee: PHARMORX INC., Wellesley, MA (US)

(21) Appl. No.: 11/458,890

(22) Filed: Jul. 20, 2006

Related U.S. Application Data
(60) Provisional application No. 60/701,013, filed on Jul. 20, 2005.

(51) Int. Cl. A61K 31/485; A61K 9/50; A61K 9/16

(52) U.S. Cl. 424/490; 514/282

(57) ABSTRACT

Pharmaceutical dosage forms are provided for use in deterring abuse of opioids or other medications, which help avoid harm to a patient dependent on the medication. In one case, a pharmaceutical oral dosage form is provided that includes a plurality of microcapsules, each microcapsule of the plurality containing an opioid agonist medication in a controlled release form, and a partial opioid agonist sequestered in the pharmaceutical dosage form, such that upon oral administration of the pharmaceutical oral dosage form the partial opioid agonist will pass through the gastrointestinal tract without uptake by the body.

### CROSS REFERENCE TO RELATED APPLICATIONS

This application claims benefit of U.S. Provisional Application No. 60/701,-13, filed Jul. 20, 2005. That application is incorporated herein by reference.

### BACKGROUND OF THE INVENTION

This invention is generally in the field of pharmaceutical formulations useful in combating abuse of medications, and more particularly to formulating medications so that an abuser is impeded from attaining a desired intense euphoria or other physiological alteration sought by the abuser.

Opioid agonists are a class of drugs used primarily as moderate to strong analgesics, but they have other pharmacological effects including euphoria and mental clouding without loss of consciousness. Because of these other pharmacological effects and the strong negative reinforcement resulting from rapid termination of physical or psychic pain, opioids have become the subject of dependence and abuse. Four high dose euphoric “hits ” are sufficient to produce opioid addiction. Thus opioids, such as morphine, hydromorphone, hydorcordone and oxycodone, are effective in the management of pain; however, there has been an increase in their abuse by individuals who are psychologically dependent on opioids or who misuse opioids for non-therapeutic reasons.

There are a variety of different approaches proposed for protecting oral opioids from abuse. For example, U.S. Pat. No. 6,696,088 to Oshlack et al. discloses a controlled released opioid analgesic, which is formulated with an opioid antagonist such as naltrexone HCl, which is itself formulated in a unique controlled release matrix. Upon conditions of misuse or tampering, the antagonist is liberated, yet released in amounts not affecting the analgesia experienced by the patient under the prescribed conditions of use. However, there is evidence that the analgesic efficacy of these combinations can be reduced.

Previous experience with opioids has demonstrated a somewhat decreased abuse potential when the opioid is administered in combination with a narcotic antagonist, especially in patients who are ex-addicts (Weinhold et al., Drug & Alcohol Dependence 30:263-74 1992); Mendelson et al., Clin. Pharm Ther. 60:105-14 (1996)). The opioid antagonist is available for release in the gastrointestinal tract when orally administered, but it is poorly absorbed unless the material is injected or inhaled.

PCT Application Publication WO 01/58451 discloses the use of a pharmaceutical composition that contains a substantially non-releasing opioid antagonists and a releasing opioid antagonist as separate subunits that are combined into a pharmaceutical dosage form, e.g., tablet or capsule. However, because the agonist and antagonist are in physically separate compartments, they can be readily separated. Further, by providing the agonist and antagonist as separate compartments, the tablets are more difficult to form, due to the mechanical sensitivity of some subunits comprising a sequestering agent.

U.S. Pat. No. 5,236,714 also discloses incorporating an opioid antagonist to block the effectiveness of an opioid agonist, especially under conditions of abuse or tampering or in the absence of medical activation. Such approaches with opioid antagonists suffer the drawback of precipitating acute opioid withdrawal in the abusing patient.

A patient who is physically dependent on opioid drugs will go into “precipitated withdrawal” and could become violently and possibly dangerously ill when the opioid antagonist is administered. Depending upon the drug upon which dependence has been established and the duration of use and dose, symptoms of withdrawal vary in number and kind, duration and severity. The most common symptoms of the opioid withdrawal syndrome include anorexia, nausea, pupillary dilation, chills alternating with excessive sweating, abdominal cramps, nausea, vomiting, muscle spasms, hyperirritability, lacrimation, rhinorrhea, goose flesh and increased heart rate. Natural abstinence syndromes typically begin to occur 24-48 hours after the terminating the opioid agonist, reach maximum intensity about the third day, and may not begin to decrease until the third week. Precipitated abstinence syndromes produced by administration of an opioid antagonist vary in intensity and duration with the dose and the specific antagonist, but generally vary from a few minutes to several hours in length. Such a patient could engage in dangerous, criminal behavior in a desperate attempt to obtain drugs to reverse the precipitated withdrawal syndrome. Furthermore, the repeated experience of withdrawal is thought to strengthen the desire to obtain opioids in the future and thereby could worsen the patient's opioid addiction.

In another stratagem, exemplified by U.S. Patent Application Publication Nos. 2003/0091635, 2004/0052731, and 2003/0118641, a relatively lipophilic delivery complex is formed around the opioid agonist, which provides slow release in vivo and makes it more difficult to illicitly extract the agent using common solvents. However, given the art of biochemistry and sufficient patience on the part of the tamperer, a chemical environment similar to the human stomach-small intestine, including purified digestive enzymes, can be created in vitro, allowing for eventual extraction of an agonist such as oxycodone. The danger of this invitation to illicit chemists is underscored by a recipe currently available on the Internet for inexpensively converting extracted oxycodone to oxymorphone, its normal metabolite which is ten times more potent and correspondingly more valuable. It therefore would be desirable to provide a dosage form wherein it is highly difficult or impossible to separate the opioid agonist from its controlled release means without causing the opioid agonist to be rendered ineffective.

Accordingly, it would be desirable to provide improved pharmaceutical dosage forms wherein the medication is effective if taken according to medical directions, and wherein the medication is ineffective taken in a manner contrary to the medical directions. Furthermore, it would be highly desirable to provide an abuse deterrent form of opioid agonists that causes no or minimal harm to a patient dependent on the medication, e.g., to avoid precipitated withdrawal, even if taken in a manner contrary to medical directions.

### SUMMARY OF THE INVENTION

Pharmaceutical dosage forms are provided for use in deterring abuse of opioids or other medications, which help avoid harm to a patient dependent on the medication. In one aspect, pharmaceutical oral dosage form is provided that includes a plurality of microcapsules, each microcapsule of the plurality containing an opioid agonist medication in a controlled release form, and a partial opioid agonist sequestered in the pharmaceutical dosage form, such that upon oral administration of the pharmaceutical oral dosage form the partial opioid agonist will pass through the gastrointestinal tract without uptake by the body.

In a preferred embodiment, the dosage form includes (1) a first plurality of microcapsules, each microcapsule of the first plurality containing an opioid agonist medication in a controlled release form, and (2) a second plurality of microcapsules, each microcapsule of the second plurality containing a partial opioid agonist, wherein the partial opioid agonist is provided in a sequestered form in the pharmaceutical dosage form, such that the partial opioid agonist is released in an effective amount in vivo if the pharmaceutical dosage form is administered to a person in an altered form but is not released in an effective amount in vivo if the pharmaceutical dosage form is administered to a person in a substantially unaltered form. In preferred embodiments, the microcapsules of the first plurality and of the second plurality have the same specific density, the same flocculation characteristics, and the same color, size, and textural appearance.

The microcapsules of the first plurality and of the second plurality preferably are mixed together within a unit dose form. The pharmaceutical dosage form may be a tablet or capsule suitable for oral administration.

The microcapsules containing the opioid agonist or other therapeutic medication may further comprise one or more matrix materials, which provide controlled release of the therapeutic medication. The matrix material may be or include a hydrophobic and/or amphiphilic compound and/or a bioerodible or biodegradable polymer.

In a preferred embodiment, the microcapsules of the second plurality include a non-degradable layer covering the partial opioid agonist. Other sequestration means may alternatively or additionally be used.

The opioid agonist medication may be oxycodone, hydromorphone, hydrocodone, or a combination thereof. The partial opioid agonist may be buprenorphine.

### BRIEF DESCRIPTION OF THE DRAWINGS

FIG. 1 is a cross-sectional view of one embodiment of a pharmaceutical dosage form as described herein.

FIG. 2 is a cross-sectional view detailing one embodiment of a first type of microcapsule in the pharmaceutical dosage form illustrated in FIG. 1.

FIG. 3 is a cross-sectional view detailing one embodiment of a second type of microcapsule in the pharmaceutical dosage form illustrated in FIG. 1.

FIG. 4 is a cross-sectional view of another embodiment of a pharmaceutical dosage form as described herein.

FIG. 5 is a cross-sectional view of another embodiment of a pharmaceutical dosage form as described herein.

FIG. 6 is a cross-sectional view of another embodiment of a pharmaceutical dosage form as described herein.

### DETAILED DESCRIPTION OF THE PREFERRED EMBODIMENTS

Pharmaceutical dosage forms and methods have been developed to thwart abuse of medications.

In one aspect, a combination of an opioid agonist first medication in timed release form, and a sequestered partial agonist medication allows the first medication to be effective if taken according to medical directions. If the combination is tampered with or otherwise taken in a manner not according to medical directions, the second medication is released, which is a partial opioid agonist binding strongly to opioid mu receptors and displacing the first agonist or other full opioid agonists from the receptors, preventing an euphoric response without precipitating opioid withdrawal. In other words, when the material of the pharmaceutical dosage form is introduced into the body in a manner unintended by the manufacturer, the partial opioid agonist strongly binds to opioid receptors, displacing other opioid agonists, but produces a blunted response—even if the medication is present in high concentration. The partial opioid agonist is not released into the person's body if the microcapsules of the second plurality are substantially intact when the unaltered dosage form is ingested.

More generally, the present pharmaceutical oral dosage forms are technology is useful for a number of other drugs where sustained release oral delivery is desired, and there is the potential for abuse if the drug dose is made immediately available for nasal, IV or oral administration. For instance, the dosage forms may include a controlled release form of another agonist medication admixed with a normally unabsorbed second medication which, if it were absorbed in the body of a patient, would prevent the desired intense euphoria or “hit”. When taken according to medical directions, the second medication is blocked from absorption in the body, and when the material is taken contrary to medical directions such as grinding or chewing the material, the second medication is released, and, especially with attempts to inject or inhale the contents, the euphoric response to the first medication or virtually any other opioid full agonist, is blocked. Because the second medication is a partial agonist, it advantageously does not typically produce withdrawal in the active abuser.

As used herein, the terms “comprise, ” “comprising,” “include, ”0 and “including” are intended to be open, non-limiting terms, unless the contrary is expressly indicated.

In one aspect, a pharmaceutical dosage form is provided which includes (1) a plurality of microcapsules, each microcapsule of the plurality containing an agonist medication in a controlled release form, and (2) a partial agonist sequestered in the pharmaceutical dosage form, such that upon oral administration of the pharmaceutical oral dosage form, in substantially unaltered form, the partial agonist will pass through the gastrointestinal tract without uptake by the body. The agonist medication can be any medicament, but preferably is one that is addictive (physically and/or psychologically) and typically leads to abuse, as manifested by taking the medication too frequently or repeatedly.

In one particular embodiment, the pharmaceutical dosage form includes a first plurality of microcapsules, each microcapsule of the first plurality containing an opioid agonist medication in a controlled release form; and a second plurality of microcapsules, each microcapsule of the second plurality containing a partial opioid agonist, wherein the partial opioid agonist is provided in a sequestered form in the pharmaceutical dosage form, such that the partial opioid agonist is released in an effective amount in vivo if the pharmaceutical dosage form is administered to a person in an altered form but is not released in an effective amount in vivo if the pharmaceutical dosage form is administered to a person in a substantially unaltered form. The microcapsules of the first plurality and of the second plurality preferably are mixed together within a unit dose form. The pharmaceutical dosage form may be a tablet or capsule suitable for oral administration.

In a preferred embodiment, when the tablet, pill, capsule, or other dosage form of the present pharmaceutical dosage forms is chewed, ground, or otherwise treated contrary to medical instructions to make the entire dose of opioid agonist medication available for immediate absorption, the normally sequestered partial agonist medication is also liberated from the sequestering means and becomes immediately available for absorption, either sublingually or especially when the contents of the altered dosage form are injected or inhaled. It is a distinctive advantage of the present dosage forms that the normally sequestered medication is not an opioid antagonist, and would not precipitate withdrawal in a dependent patient. In fact, it is now clear that addicted individuals anticipating withdrawal may seek out the preferred embodiment partial agonist, i.e., buprenorphine, for its prevention, but they do not derive a euphoric response from it or other opioids taken shortly thereafter.

Microcapsules

As used herein, the term “microcapsules” includes microparticles, microspheres, and may or may not be spherical in shape. The microcapsules may or may not have an outer polymer shell surrounding a core of active agent. They may be solid spheres, which can include a honeycombed structure formed by pores through the polymer which are filled with the active agent. The microcapsules typically have a diameter of between 0.5 and 5 mm.

Opioid Agonist Medication

As used herein, the term “opioid” is meant to include a drug, hormone, or other chemical or biological substance, natural or synthetic, having a sedative, narcotic, or otherwise similar effect(s) to those containing an opium or its natural or synthetic derivatives. By “opioid agonist,” and “agonist” sometimes used herein interchangeably with terms “opioid” and “opioid analgesic,” is meant to include one or more opioid agonists, either alone or in combination, and its further meant to include the base of the opioid, mixed or combined agonist-antagonists, pharmaceutically acceptable salts thereof, stereoisomers thereof, ethers thereof, esters thereof, and combinations thereof.

Representative examples of opioid agonists include alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, proheptazine, promedol, properidine, propiram, propoxyphene, sufentanil, tilidine, tramadol, pharmaceutically acceptable salts thereof, and mixtures thereof.

In preferred embodiments, the opioid agonist is selected from codeine, hydromorphone, hydrocodone, oxycodone, dihydrocodeine, dihydromorphine, morphine, tramadol, oxymorphone, pharmaceutically acceptable salts thereof, and mixtures thereof.

Effective amounts of the therapeutic agents are known or can be readily ascertain by those skilled in the art.

Controlled Release Form

The opioid agonist preferably is provided in a controlled release form. For example, the controlled release component of the microcapsules of the first plurality of microcapsules may include one or more biocompatible matrix materials. As used herein, the term “matrix” refers to a structure including one or more materials in which a drug is dispersed, entrapped, or encapsulated. Such matrix materials are known in the art. For example, the matrix material may comprise a hydrophobic and/or amphiphilic compound. In one case, the matrix material may comprise a biocompatible, bioerodible or biodegradable polymer. In another example, the controlled release form may include dendritic polymer formulations. In still another example, the dosage form comprises an opioid agonist dispersed in a matrix which comprises a polymeric material and a hydrophobic and/or amphiphilic compound that modifies the period of drug release as compared with the same polymeric matrix without the incorporated hydrophobic compound, by altering the rate of diffusion of water into and out of the matrix and/or the rate of degradation of the matrix. In yet another example, controlled release of the opioid agonist utilizes liposome structures.

In some embodiments, the individual drug-containing microcapsules or opioid agonist particles are coated with one or more independent coating layers. At least one of the coating materials is water-insoluble and preferably organic solvent-insoluble, but enzymatically degradable. Desirably, the components of the coated microparticles are not mutually soluble in water, organic solvents, or a combination thereof, so that in vitro degradation of the formulation will require more than one step, such that extraction of the opioid agonist is not easily accomplished using conventional chemical means. In contrast, when administered to the gastrointestinal tract via swallowing, the opioid agonist gradually will be released from the coated microcapsules as a consequence of enzymatic degradation, surfactant action of bile acids, and mechanical erosion within the GI tract.

Partial Opioid Agonist

The partial opioid agonist ideally is selected for strong binding (to mu opioid receptors) sufficient to displace opioid full agonists. Unlike the case with agonists, with the “partial agonist”, despite increasing concentrations of the agent, the effect is not full, i.e., no euphoria. A morphine-like structure would make chemical separation from common opioid agonists more difficult. This partial opioid agonist, which binds strongly to opioid mu receptors, would displace virtually any other opioid, but, by definition of a partial agonist, produce only a blunted or partial response even at high concentration. The partial opioid agonist is not an antagonist such as naloxone or naltrexone, and, unlike these compounds, it would not typically produce withdrawal.

The most extensively characterized currently utilized opioid partial agonist in medical practice (for treatment of opioid addiction) is buprenorphine. Accordingly, the partial opioid agonist preferred in the present dosage form is buprenorphine. However, given the clear pharmacologic definitions of an opioid partial agonist and strong binding (low Ki) to u receptors, those with experience in the art of pharmacology could discover or synthesize alternative protective agents for sequestration.

Optimal dosages of the sequestered partial agonist will be based on emetic properties (desirable for disincentive to abuse and protection against opioid overdose), absorption from the gastrointestinal tract, and safety when injected or inhaled. Dose determinations are readily established by those with knowledge of the art of pharmacology.

It is noted that buprenorphine has a morphine-like structure, and is therefore difficult to separate chemically from preferred opioid agonists. Moreover, because it binds at least 1000 times more tightly to mu receptors than oxycodone, despite its lipophilicity and oxycodone's hydrophilicity, extraction by water or alcohol from a tampered vehicle will not defeat the protective effect of buprenorphine with oxycodone. The protection of other many of opioid agonists is even better.

Sequestration of the Partial Opioid Agonist

A wide range of techniques, structures, and materials in various combinations may be used to sequester the partial opioid agonist. Many suitable sequestration techniques are known in the art. Essentially any of the available technologies for sequestering a second medication from a normal digestion may be employed. In one embodiment, buprenorphine or another partial opioid agonist is incorporated in one or more discrete compartments within the dosage form, sequestered from digestion, which would be released only upon tampering, and would then displace the time release agonist and other agonists being abused from receptors, and, in attempts to chemically extract the agonist, would frustrate purification or chemical production of more potent agonists.

In one embodiment, the microcapsules of the second plurality comprise a non-degradable layer covering the partial opioid agonist. As used herein, the term “non-degradable” refers to a material that is substantially insoluble in fluids of the gastrointestinal tract of a human and substantially non-erodible passing through the gastrointestinal tract. The non-degradable layer is also preferably substantially non-porous.

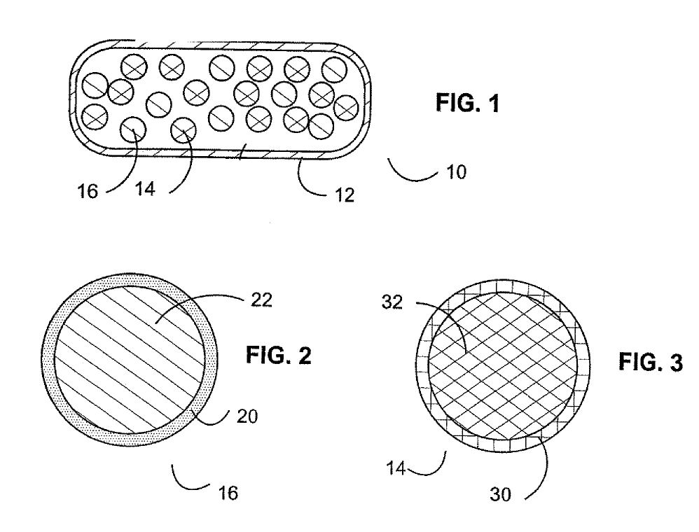

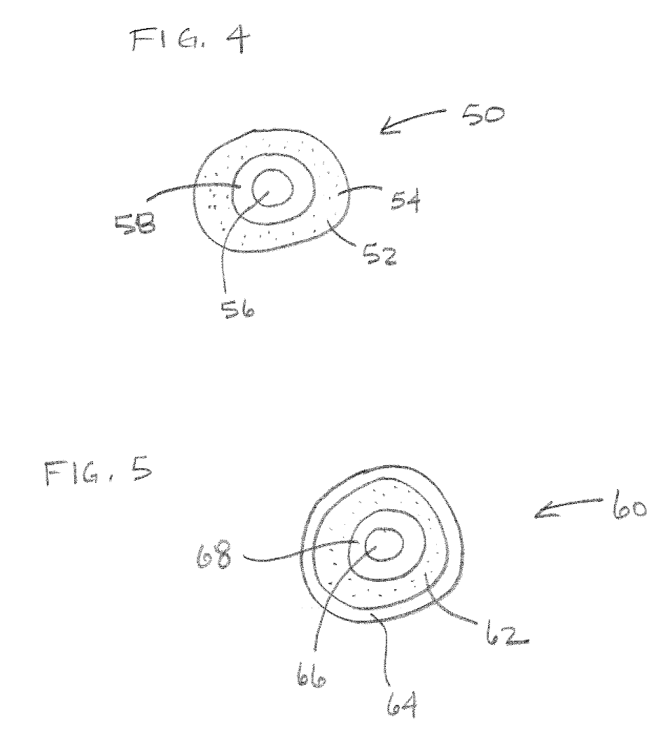

FIG. 1 illustrates a dosage form 10 (e.g., capsule, pill, or tablet) 10 which includes firs microcapsules 14 and second microcapsules 16, encased in optional outer covering 12 (e.g., a capsule). FIG. 2 shows microcapsules 16 in more detail. Outer coating 20 encapsulates the first opioid agonist medication 22. The outer coating 20 dissolves in vivo (preferably in the gastrointestinal tract) and controllably releases the opioid agonist medication 22. Depending on the particular outer coating, the medication 22 may be released faster or slower than the release of medication 22 from other microcapsules 16. For example, different materials or coating thicknesses could be employed. In other embodiments, control of release of medication 22 may use alternative techniques known in the art for controlled release. FIG. 3 shows microcapsules 14 in more detail. The outer coating 30 of microcapsule 14 is normally not dissolved in the digestive system of the patient, and the medication 32 passes through the digestive system with no effect. However, if the capsule 10 is ground, chewed, or otherwise tampered with, the medication 32 is released and comingled with the first medication.

In a preferred embodiment, to deter improper separation of the microcapsules 14 from microcapsules 16, microcapsules 14 and 16 have the same specific density, so that separating the microcapsules by settling or centrifuging is hindered. Similarly, microcapsules 14 and 16 preferably have coatings which have similar flocculation properties, so that separation by flocculation is hindered. In addition, microcapsules 14 and 16 preferably look identical. That is, they may have coatings which have similar visual appearance, color, size, and texture, again to hinder separation by optical, sizing, or other separation techniques known in or readily adapted from the art. This may be achieved by coating both microcapsules 14 and 16 with the top coating layer material that rapidly dissolves or degrades in vivo so as not to impede operation of the controlled release means for controlling release of the opioid agonist medication.

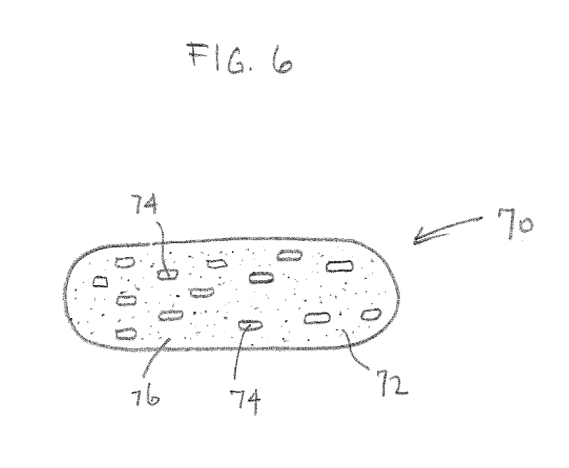

In another embodiment, both the opioid agonist and the partial agonist are contained together within a single structure, e.g., a microcapsule or tablet, in separate regions, to achieve the abuse-resistance characteristics provided by the two-microcapsule-types approach described above. Examples of such dosage form structures are illustrated in FIGS. 4-5.

In FIG. 4, microcapsule 50 includes a core of partial opioid agonist 56 which is covered by non-degradable covering 58. This covered core is surrounded by an opioid agonist 52 dispersed in a controlled release matrix material 54. If the microcapsule 50 is crushed by a tamperer in an effort to free the opioid agonist 52, the covering 58 will also be ruptured to release the partial opioid agonist 56. If used as intended, the opioid agonist will be released in a controlled manner in the gastrointestinal (GI) tract following oral administration and the covered core will pass through the GI tract without releasing the partial opioid against 56.

The microcapsule 60 shown in FIG. 5 functions similarly, but has a different controlled release structure than that of microcapsule 50. It includes a core of partial opioid agonist 66 which is covered by non-degradable covering 68. This covered core is surrounded by an opioid agonist 52 which is in turn covered by a separate layer of a controlled release matrix material 54.

In one embodiment, the dosage form is prepared by solid free form fabrication methods, which are described for example in U.S. Pat. No. 6,280,771, which is incorporated herein by reference. For example, a tablet may be constructed in multiple layers, with various concentration gradients or other variations in micro-architectures. Polymer shell materials can be includes with varying thicknesses, saturation levels, densities, etc. to further control release kinetics. In one example, three dimensional printing may be used to build the dosage form, wherein the opioid agonist to be delivered and the sequestered opioid partial agonist “randomly” placed in different, but discrete chambers/regions of the dosage form, making the dosage form more difficult to reverse engineer. FIG. 6 illustrates an example of a tablet 70 that includes discrete regions of sequestered partial agonist 74 which are randomly located within a controlled release matrix material 76. The opioid agonist 72 is also dispersed in the controlled release matrix material 76.

Dosage Forms and Optional Components

The present compositions/dosages forms can optionally comprise a suitable amount of a pharmaceutically acceptable vehicle so as to provide the form for proper administration to the patient. As used herein, the term “pharmaceutically acceptable” means approved by a regulatory agency of the Federal or a state government or listed in the U.S. Pharmacopeia or other generally recognized pharmacopeia for use in animals, mammals, and more particularly in humans. The term “vehicle” refers to a diluent, adjuvant, excipient, or carrier with which a pharmaceutically active ingredient is administered. Such pharmaceutical vehicles are well known in the art. The vehicle may include bulking agents, stabilizers, thickeners, wetting agents, emulsifiers, pH buffers, lubricants, colorants, taste masking agents, and the like, as known in the art.

In various embodiments, the dosage form may be a tablet, pill, pellet, capsule, suspension, powder, suppository, or other suitable form. In a preferred embodiment, the dosage form is formulated in accordance with routine procedures as a pharmaceutical composition adapted for oral administration to human beings. Compositions for oral delivery may be in the form of tablets, lozenges, aqueous or oily suspensions, granules, powders, emulsions, capsules, syrups, or elixirs, for example. Orally administered compositions may contain one or more agents, for example, sweetening agents such as fructose, aspartame or saccharin; flavoring agents; coloring agents; and preserving agents, to provide a pharmaceutically palatable preparation. Moreover, where in tablet or pill form, the compositions can be coated to delay disintegration and absorption in the gastrointestinal tract, thereby providing a sustained action over an extended period of time. Selectively permeable membranes surrounding an osmotically active driving compound are also suitable for orally administered compositions. In these later platforms, fluid from the environment surrounding the capsule is imbibed by the driving compound, which swells to displace the agent or agent composition through an aperture. A time delay material such as glycerol monostearate or glycerol stearate may also be used. Oral compositions can include standard vehicles such as pharmaceutical grade mannitol, lactose, starch, magnesium stearate, sodium saccharin, cellulose and magnesium carbonate.

The pharmaceutical dosage form is preferably in unit dosage form. In this way, the preparation is subdivided into unit doses containing appropriate quantities of the opioid agonist. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

To increase the deterrent effect of the present pharmaceutical dosage forms, in one embodiment, a pill form may be packaged with an instruction that biting or chewing the pill may produce nausea or vomiting, and that the contents of the pill will be ineffective if injected or inhaled.

Publications cited herein are incorporated by reference. Modifications and variations of the methods and devices described herein will be obvious to those skilled in the art from the foregoing detailed description. Such modifications and variations are intended to come within the scope of the appended claims.

I claim:

1. A pharmaceutical dosage form comprising:
a first plurality of microcapsules, each microcapsule of the first plurality containing an opioid agonist medication in a controlled release form; and
a second plurality of microcapsules, each microcapsule of the second plurality containing a partial opioid agonist,
wherein the partial opioid agonist is provided in a sequestered form in the pharmaceutical dosage form, such that the partial opioid agonist is released in an effective amount in vivo if the pharmaceutical dosage form is administered to a person in an altered form but is not released in an effective amount in vivo if the pharmaceutical dosage form is administered to a person in a substantially unaltered form.

2. The pharmaceutical dosage form of claim 1, wherein the opioid agonist medication comprises oxycodone, hydromorphone, hydrocodone, or a combination thereof.

3. The pharmaceutical dosage form of claim 1, wherein the partial opioid agonist comprises buprenorphine.

4. The pharmaceutical dosage form of claim 1, wherein the microcapsules of the first plurality and of the second plurality have the same specific density.

5. The pharmaceutical dosage form of claim 1, wherein the microcapsules of the first plurality and of the second plurality have the same flocculation characteristics.

6. The pharmaceutical dosage form of claim 1, wherein the microcapsules of the first plurality and of the second plurality have the same color, size, and textural appearance.

7. The pharmaceutical dosage form of claim 1, wherein the microcapsules of the first plurality further comprise one or more matrix materials.

8. The pharmaceutical dosage form of claim 7, wherein matrix material comprises a hydrophobic and/or amphiphilic compound.

9. The pharmaceutical dosage form of claim 7, wherein matrix material comprises a bioerodible or biodegradable polymer.

10. The pharmaceutical dosage form of claim 1, which comprises a tablet or capsule suitable for oral administration.

11. The pharmaceutical dosage form of claim 1, wherein the microcapsules of the second plurality comprise a non-degradable layer covering the partial opioid agonist.

12. The pharmaceutical dosage form of claim 1, wherein the microcapsules of the first plurality and of the second plurality are mixed together within a unit dose form.

13. A pharmaceutical oral dosage form comprising:
a plurality of microcapsules, each microcapsule of the plurality containing an opioid agonist medication in a controlled release form; and
a partial opioid agonist sequestered in the pharmaceutical dosage form, such that upon oral administration of the pharmaceutical oral dosage form the partial opioid agonist will pass through the gastrointestinal tract without uptake by the body.

14. The pharmaceutical oral dosage form of claim 13, wherein the first opioid agonist medication comprises oxycodone, hydromorphone, hydrocodone, or a combination thereof.

15. The pharmaceutical dosage form of claim 14, wherein the partial opioid agonist comprises buprenorphine.

16. The pharmaceutical dosage form of claim 13, wherein the partial opioid agonist is covered by a non-degradable layer.

17. A pharmaceutical oral dosage form comprising:
opioid agonist medication particles coated by or dispersed in a degradable matrix material which provides controlled release in vivo; and
partial opioid agonist coated by or dispersed in a non-degradable material.
